(19) United States
(12) Patent Application Publication
(10) Pub. No.: US 2009/0131635 A1
(43) Pub. Date: May 21, 2009

(54) Method for synthesizing polymer on substrate

(76) Inventors: Sung-Min Chi, Hwaseong-si (KR); Man-Hyoung Ryoo, Hwaseong-si (KR); Jung-Hwan Hah, Hwaseong-si (KR); Kyoung-Seon Kim, Suwon-si (KR); Won-Sun Kim, Suwon-si (KR); Sun-Ok Jung, Suwon-si (KR)

Correspondence Address: LAW OFFICE OF MONICA H CHOI, P O BOX 3424, DUBLIN, OH, 430160204, US

(21) Appl. No.: 12/220,923

(22) Filed: Jul. 30, 2008

(30) Foreign Application Priority Data
Jul. 30, 2007 (KR) 10-2007-0076518

(51) Int. Cl. C07K 1/04; C07H 1/00

(52) U.S. Cl. 530/333; 536/25.3

(57) ABSTRACT

For synthesizing a polymer, a substrate is placed within a reaction chamber, and a polymer synthesis sample is fed into the reaction chamber for forming the polymer on the substrate. In addition, the reaction chamber is shaken during formation of the polymer on the substrate within the reaction chamber for increased reaction yield. In addition, forming bubbles of the inactive gas in the polymer synthesis sample during formation of the polymer on the substrate further increases reaction yield.

This application claims priority under 35 USC § 119 to Korean Patent Application No. 10-2007-0076518, filed on Jul. 30, 2007 in the Korean Intellectual Property Office, the disclosure of which is incorporated herein in its entirety by reference.

### BACKGROUND OF THE INVENTION

1. Field of the Invention

The present invention relates generally to synthesis of polymer on a substrate, and more particularly to shaking a reaction chamber during synthesis of the polymer for improving reaction yield.

2. Background of the Invention

Synthesis of polymers on a substrate is increasingly desired in various fields including semiconductors. For example, micro-arrays having biopolymers such as oligomer probes fixed onto a slide substrate have been introduced in recent years. Polymer synthesis technology is also employed to form such micro-arrays.

A photolithographic technique widely used in semiconductor fabrication may be applied to synthesize oligomer probes in a micro-array. Such synthesis of oligomer probes using photolithography includes attaching a coupling agent containing a photo-labile protecting group onto a substrate, removing the photo-labile protecting agent from the coupling agent after selective exposure through a photo-mask, and providing a monomer to be synthesized onto the exposed coupling agent.

To synthesize 25-mer oligomer probes, the synthesis step is repeated 25 to 100 times. A reaction yield at each step between a monomer to be synthesized and a coupling agent may significantly affect the overall processing yield. Thus, maximizing the reaction yield at each synthesis step is desired.

### SUMMARY OF THE INVENTION

Accordingly for synthesizing a polymer, a substrate is placed within a reaction chamber, and a polymer synthesis sample is fed into the reaction chamber for forming the polymer on the substrate. In addition, the reaction chamber is shaken during formation of the polymer on the substrate within the reaction chamber.

In an example embodiment of the present invention, the reaction chamber includes a chamber body and a chamber cover that is combined with the chamber body to form a sealed reaction space.

In another embodiment of the present invention, the reaction space is sealed with an edge of the substrate abutting a rim of one of the chamber body or the chamber cover.

In an embodiment of the present invention, the reaction space is formed between the chamber cover and one surface of the substrate when the edge of the substrate abuts the rim of the chamber cover. In addition, an air space is formed between the chamber body and another surface of the substrate. In that case, the reaction space is sealed from the air space.

In another embodiment of the present invention, another surface of the substrate abuts the chamber body.

In a further embodiment of the present invention, the chamber cover includes a transparent portion for viewing into the reaction space.

In another embodiment of the present invention, the chamber cover includes at least one fluid inlet/outlet. In that case, the polymer synthesis sample is fed into the reaction space through the at least one fluid inlet/outlet. In addition, an inactive gas is fed into the reaction chamber through the at least one fluid inlet/outlet for generating bubbles in the polymer synthesis sample within the reaction chamber during formation of the polymer. Furthermore, the polymer synthesis sample is drained through the at least one fluid inlet/outlet after formation of the polymer on the substrate.

In a further embodiment of the present invention, a distance of the reaction space between the chamber cover and the surface of the substrate is in a range of from about 0.2 mm (millimeters) to about 10 mm (millimeters).

In another embodiment of the present invention, the reaction space is formed between the chamber body and one surface of the substrate.

According to another aspect of the present invention, the polymer synthesis sample fills from about 10% to about 90% of the reaction space as the reaction chamber is shaken during formation of the polymer on the substrate. For example, the polymer synthesis sample fills about 60% of the reaction space as the reaction chamber is shaken during formation of the polymer on the substrate.

In a further embodiment of the present invention, the step of shaking the reaction chamber includes rolling the reaction chamber with a maximum angle in a range of from about ±10° to about ±60°. For example, the reaction chamber rolls with the maximum angle of about ±30°.

In an example embodiment of the present invention, the polymer is a biopolymer that is one of a nucleoside, a nucleotide, an amino acid, or a peptide, and the substrate is one of a semiconductor wafer or a glass substrate.

According to another aspect of the present invention, a biopolymer synthesis apparatus for synthesizing a biopolymer includes a reaction chamber including a substrate on which a biopolymer is to be synthesized, and includes a shaking unit for shaking the reaction chamber.

In this manner, with shaking of the reaction chamber, reaction yield is increased for the polymer formed on the substrate. In addition, forming bubbles of the inactive gas in the polymer synthesis sample during formation of the polymer on the substrate further increases reaction yield.

### BRIEF DESCRIPTION OF THE DRAWINGS

The above and other features and advantages of the present invention will become more apparent when described in detailed exemplary embodiments thereof with reference to the attached drawings in which:

FIG. 1 is a top view of a biopolymer synthesis apparatus, according to an embodiment of the present invention;

FIG. 2 is a front view of the biopolymer synthesis apparatus of FIG. 1, according to an embodiment of the present invention;

FIG. 3 is a perspective view of the reaction chamber of FIG. 1, according to an embodiment of the present invention;

FIGS. 4A and 4B are cross-sectional views of the reaction chamber of FIG. 1, according to an embodiment of the present invention;

FIG. 5 is a cross-sectional view of the reaction chamber of FIG. 1, according to another embodiment of the present invention;

FIG. 6 is a schematic diagram of a fluid flow system for the biopolymer synthesis apparatus, according to an embodiment of the present invention;

FIG. 7 is a side view illustrating a shaking of the reaction chamber in the biopolymer synthesis apparatus of FIG. 1, according to an embodiment of the present invention;

FIGS. 8, 9, 10, and 11 are cross-sectional views illustrating the synthesis of a biopolymer within the reaction chamber in FIG. 1, according to an embodiment of the present invention;

FIGS. 12A, 12B, 12C, and 12D are cross-sectional views of the substrate within the reaction chamber of FIG. 1 for synthesis of the biopolymer, according to an embodiment of the present invention; and

FIG. 13 is a flowchart of steps for synthesis of the biopolymer on the substrate within the reaction chamber of FIG. 1, according to an embodiment of the present invention.

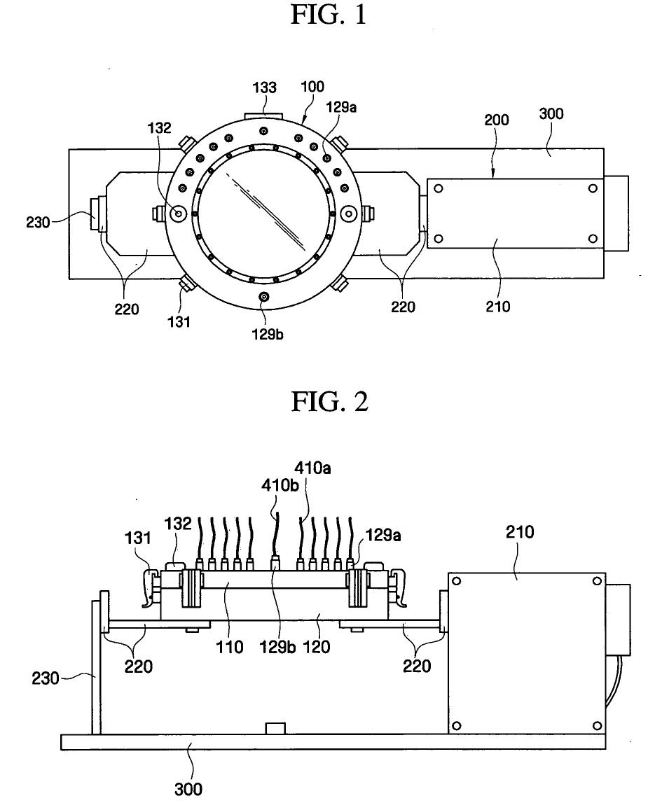

The figures referred to herein are drawn for clarity of illustration and are not necessarily drawn to scale. Elements having the same reference number in FIGS. 1, 2, 3, 4A, 4B, 5, 6, 7, 8, 9, 10, 11, 12A, 12B, 12C, 12D, and 13 refer to elements having similar structure and/or function.

### DETAILED DESCRIPTION OF THE INVENTION

Advantages and features of the present invention and methods of accomplishing the same may be understood more readily by reference to the following detailed description of preferred embodiments and the accompanying drawings. The present invention may, however, be embodied in many different forms and should not be construed as being limited to the embodiments set forth herein. Rather, these embodiments are provided so that this disclosure will be thorough and complete and will fully convey the concept of the invention to those skilled in the art.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural-forms as well as the singular forms, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

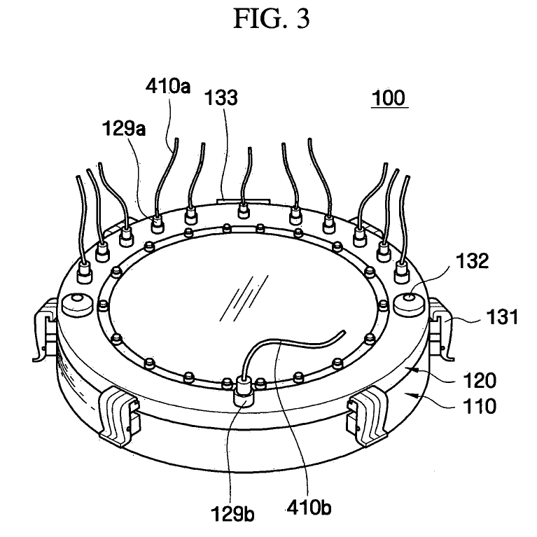

FIG. 1 is a top view of a biopolymer synthesis apparatus according to an embodiment of the present invention. FIG. 2 is a front view of the biopolymer synthesis apparatus of FIG. 1. FIG. 3 is a perspective view of a reaction chamber in the biopolymer synthesis apparatus of FIG. 1.

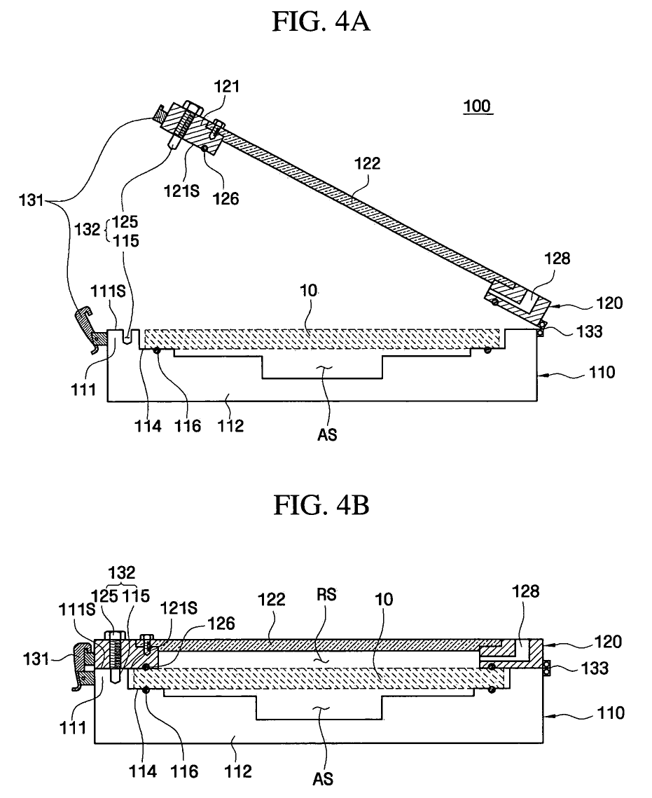

Referring to FIGS. 1, 2, and 3, the biopolymer synthesis apparatus includes a reaction chamber 100 and a shaking unit 200. The reaction chamber 100 holds a substrate (10 in FIGS. 4A and 4B) on which at least one polymer such as a biopolymer for example is to be synthesized.

Such a target biopolymer to be synthesized on the substrate includes a polymer that is typically synthesized within or that typically constitutes a living body. For example, such a biopolymer is comprised of two or more monomers with some examples of the monomers being a nucleoside, a nucleotide, an amino acid, and a peptide.

The nucleoside and nucleotide contain not only known purine and pyrimidine bases but also methylized purine or pyrimidine and acylated purine or pyrimidine. Further, the nucleoside and nucleotide may contain a conventional ribose and deozyribose sugar as well as a modified sugar formed from substituting a halogen atom or aliphatic for at least one hydroxyl group or by grafting functional groups such as ether and amine to the hydroxyl group.

The amino acid may be a D-, L- or non-chiral amino acid that may be found in nature, a modified amino acid, or an amino acid analog. The peptide includes compounds produced by a link between a carboxyl group of one amino acid and an amino group of another amino acid.

Within the reaction chamber 100, the target biopolymer is synthesized by sequentially forming a covalent bond between monomeric units on the substrate 10. Alternatively, the target biopolymer may be synthesized by creating a covalent bond between a biopolymer formed from at least two monomers covalently bonded together and another monomer or biopolymer on the substrate 10.

The substrate 10 is a base substrate that may be either flexible or rigid. For example, a flexible substrate may be a membrane such as a nylon or a Nitro cellulose (NC) or a plastic film. Alternatively, a rigid substrate may be a semiconductor wafer substrate or a transparent glass substrate such as soda-lime glass. For effective biopolymer synthesis, a monomer, a biopolymer, or other organic or inorganic linker may be fixed on the substrate.

The shape and size of the reaction chamber 10 varies depending on the shape of the substrate to be placed therein. For example, if the substrate is a circular silicon wafer, the reaction chamber 100 has a cylindrical shape.

Referring to FIGS. 1, 2, and 3, the reaction chamber 100 includes a chamber body 110 and a chamber cover 120. The chamber cover 120 is engageably combined with the chamber body 110. “Engageably combined” as used herein means that the chamber cover 120 may be completely or partially separable from the chamber body 110.

If the chamber cover 120 is at least partially separated from the chamber body 110, the interior space of the reaction chamber 100 is open for easy entry of the substrate 10 into the reaction chamber 100. When the chamber cover 120 is engaged with the chamber body 110, the interior space of the reaction chamber 100 is substantially sealed to result in a sealed reaction space RS within the reaction chamber 100 for achieving reliable reaction process control. “Substantially sealed” as used herein means the space is completely isolated from the outside, except for a controllable pipe or hole formed into the space.

The chamber body 110 is engaged with the chamber cover 120 by a first coupling unit 131 which is a clamp in the embodiment of FIGS. 1, 2, and 3. A plurality of clamps may be arranged along an outer circumference of the chamber body 110 and/or the chamber cover 120. However, the first coupling unit 131 is not limited thereto, and may have various other configurations. That is, the number and structure of the first coupling unit 131 may vary depending on the type of application. In a further exemplary embodiment of the present invention, a connecting pin 133 is located along a rim of the chamber body 110 and the chamber cover 120.

A detailed configuration of the reaction chamber 100 including its inner structure is described with reference to FIGS. 4A and 4B. FIGS. 4A and 4B are cross-sectional views of the reaction chamber 100 according to an embodiment of the present invention. FIG. 4A illustrates the reaction chamber 100 with the chamber cover 120 separated from the chamber body 110. FIG. 4B illustrates the reaction chamber 100 with the chamber cover 120 engageably combined with the chamber body 110.

Referring to FIGS. 4A and 4B, the chamber body 110 has a step height difference between a rim 111 and a central portion 112. The chamber cover 120 also has a step height difference between a rim 121 and a central portion 122. That is, the chamber body 110 and the chamber cover 120 have the rims 111 and 121, respectively, protruding higher than the central portions 112 and 122, respectively, for forming the shape of a container.

The rims 111 and 121 of the chamber body 110 and the chamber cover 120, respectively, have substantially planar surfaces 111s and 121s, respectively. Thus, when the chamber body 110 is engageably combined with the chamber cover 120, the surface 111s of the rim 111 of the chamber body 110 comes into contact with the surface 121s of the rim 121 of the chamber cover 121 while the central portion 112 of the chamber body 110 is spaced apart from the central portion 122 of the chamber cover 120.

According to an exemplary embodiment of the present invention, the reaction chamber 100 further includes second coupling units 132 disposed on the rims 111 and 121 of the chamber body 110 and the chamber cover 120. For example, the second coupling unit 132 includes a screw 125 projecting from the rim 121 of the chamber cover 120 and includes a fitting hole 115 recessed into the rim 111 of the chamber body 110. The second coupling unit 132 and the first coupling unit 131 provide precise and stable coupling between the chamber body 110 and the chamber cover 120 without misalignment.

A substrate seating surface 114 is disposed inside the rim 111 of the chamber body 110. As illustrated in FIGS. 4A and 4B, the substrate seating surface 114 has a predetermined width radially inward from the rim 111. The central portion 112 of the chamber body 110 is disposed further radially inward than the substrate seating surface 114. The central portion 112 has a larger step height difference from the rim 111 than the substrate seating surface 114.

In that case, an air space (AS) is defined between the central portion 112 of the chamber body 110 and the substrate 10 supported by the substrate seating surface 114. The substrate 10 has an edge disposed on the substrate seating surface 114 and a central portion not touching the chamber body 110.

In an alternative embodiment of the present invention, the substrate seating surface 114 may be planar across the entire central portion 112 of the chamber body 110. In that case, the entire rear surface of the substrate 10 touches and is supported by the substrate seating surface.

In another exemplary embodiment of the present invention, the rim 121 of the chamber cover 120 is radially wider than the rim 111 of the chamber body 110. In that case, when the chamber cover 120 is engageably combined with the chamber body 110, the rim 121 of the chamber cover 120 projects radially inward more than the rim 111 of the chamber body 110 such that a portion of the rim 121 of the chamber cover 120 overlaps with the substrate seating surface 114 of the chamber body 110.

Thus, if the height of the rim 111 projecting from the substrate seating surface 114 is substantially equal to the thickness of the substrate 10, an outer edge of the substrate 10 contacts the surface 121s of the rim 121 of the chamber cover 120. The outer edge of the substrate 10 serves as a sacrificial area in which biopolymer synthesis does not take place. Even though the sacrificial area with a smaller radial width is advantageous for a higher process yield, the sacrificial area preferably has a sufficient radial width to prevent contamination of a rear surface of the substrate 10, described in more detail later herein. For example, the outer edge of the substrate 10 that is touched by the rim 121 of the chamber cover 120 to form a seal has a radial width of from about 1 mm (millimeter) to about 20 mm (millimeters).

The rear surface of the substrate 10 is the surface facing the chamber body 110 in FIGS. 4A and 4B, and the rear surface of the substrate 10 would not have the biopolymer formed thereon. The front surface of the substrate 10 is the surface facing the chamber cover 120 in FIGS. 4A and 4B, and the biopolymer is to be formed on the front surface of the substrate 10.

The substrate 10 supported by the substrate seating surface 114 is spaced apart from the central portion 122 of the chamber cover 120 by the height of the rim 121 of the chamber cover 120 projecting out from the central portion 122. Such a space between the front surface of the substrate 10 and the central portion 122 of the chamber cover 120 forms a reaction space RS within which biopolymers are to be synthesized.

More specifically, the reaction space RS is defined by the central portion 122 of the chamber cover 120, a side wall of the rim 121 of the chamber cover 120, and the front surface of the substrate 10. For providing a view into the reaction chamber RS, at least the central portion 122 of the chamber cover 120 is formed of a transparent material such as glass or quartz to form a window as illustrated in FIGS. 1, 2, and 3.

The size (i.e., volume) of the reaction space RS determines the amount of a biopolymer synthesis sample used for synthesis of biopolymers on the substrate 10 and spreadability and wettability of such a sample. Such a volume of the reaction space RS depends on a distance between the central portion 122 of the chamber cover 120 and the front surface of the substrate 10 (i.e., the height of the rim 121 protruding from the central portion 122 of the chamber cover 120). In an example embodiment of the present invention, the distance between the central portion 122 of the chamber cover 120 and the front surface of the substrate 10 (or the height of the rim 121) is from about 0.2 mm to about 10 mm.

As described above, the reaction space RS is sealed within the reaction chamber 100 to be substantially isolated from the outside. The outer edge of the front surface of the substrate 10 is sealed by the rim 121 of the chamber cover 121, and the outer edge of the rear surface of the substrate 10 is supported by the substrate seating surface 114. Thus, an air space AS between the rear surface of the substrate 10 and the central portion 112 of the chamber body 110 is spatially separated from the reaction space RS. That is, the reaction space RS is substantially sealed from the air space AS.

Accordingly, when a biopolymer synthesis sample is fed into the reaction space RS, such a biopolymer synthesis sample does not infiltrate onto the rear surface of the substrate 10 for preventing contamination of the rear surface of the substrate 10. Prevention of contamination of the rear surface of the substrate 10 is desired because such contamination may cause error in analysis of biomaterials or in malfunction of a photolithography apparatus that is subsequently used.

For further ensuring prevention of contamination of the rear surface of the substrate 10 according to an alternative embodiment of the present invention, the reaction chamber 100 further includes a gasket disposed along the substrate seating surface 114 of the chamber body 110 and/or along the rim 121 of the chamber cover 120. For example, O-rings 116 and 126 are formed as the gasket in the substrate seating surface 114 and the rim 121, respectively. The O-rings 116 and 126 are in direct contact with the rear and front surfaces of the substrate 10, respectively, thus reliably preventing infiltration of a fluid between the reaction space RS and the air space AS.

Referring to FIGS. 1, 2, 3, 4A, and 4B, the reaction space RS is spatially connected to at least one fluid inlet 410a and at least one fluid outlet 410b. Alternatively, the reaction space RS is spatially connected to at least one fluid inlet/outlet when such an opening is used as both an inlet and an outlet. At least one of the chamber body 110 and the chamber cover 120 includes a plurality of openings such as through holes 128, each being coupled to at least one of the fluid inlet 410a and the fluid outlet 410b.

FIGS. 4A and 4B illustrate an example in which the through hole 128 is formed within the chamber cover 120. The through hole 128 has one end that opens at a sidewall of the rim 121 of the chamber cover 120 and the other end coupled to the fluid inlet 410a and the fluid outlet 410b through connectors 129a and 129b, respectively, as shown in FIGS. 2 and 3. A biopolymer synthesis sample, an activator, and an inactive gas are fed into (or discharged out of) the reaction space RS through the fluid inlet 410a (or fluid outlet 410b) and the corresponding through hole. Some of the fluid inlets 410a may be supply pipes dedicated for a bubble-generating inactive gas.

The operation and structure of the fluid inlet 410a and the fluid outlet 410b will be described in more detail later.

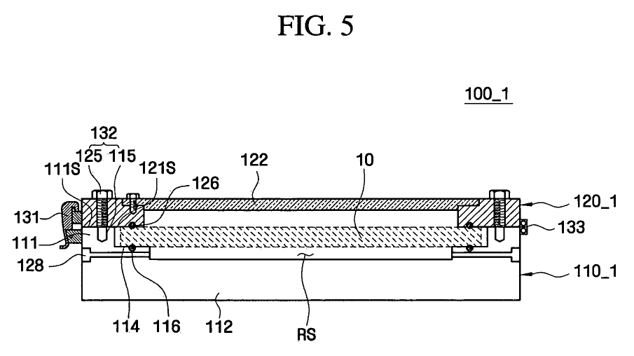

FIG. 5 is a cross-sectional view of a reaction chamber 100_1 according to another embodiment of the present invention. Elements having the same reference number in FIGS. 4B and 5 refer to elements having similar structure and/or function.

However referring to FIG. 5, the substrate 10 is turned upside down such that the reaction space RS is formed between the front surface of the substrate 10 and the central portion 112 of a chamber body 110_1. To this end, the substrate seating surface 114 of the chamber body 110_1 has the predetermined step height difference from the central portion 112 of the chamber body 110_1. The central portion 112 of the chamber body 110_1 is disposed radially inward from the substrate seating surface 114.

A distance between the central portion 112 of the chamber body 110_1 and the front surface of the substrate 10 significantly determines the size (i.e., volume) of the reaction space RS. The distance between the central portion 112 of the chamber body 110_1 and the substrate 10 is substantially equal to the height of the substrate seating surface 114 from the central portion 112 of the chamber body 110_1. Similarly, as illustrated in FIGS. 4A and 4B, the height of the substrate seating surface 114 in FIG. 5 is in a range of from about 0.2 to about 10 mm. Because the reaction space RS is defined by the chamber body 110_1, the chamber body 110_1 includes a plurality of through holes 128 connected to at least one fluid inlet and/or at least one fluid outlet.

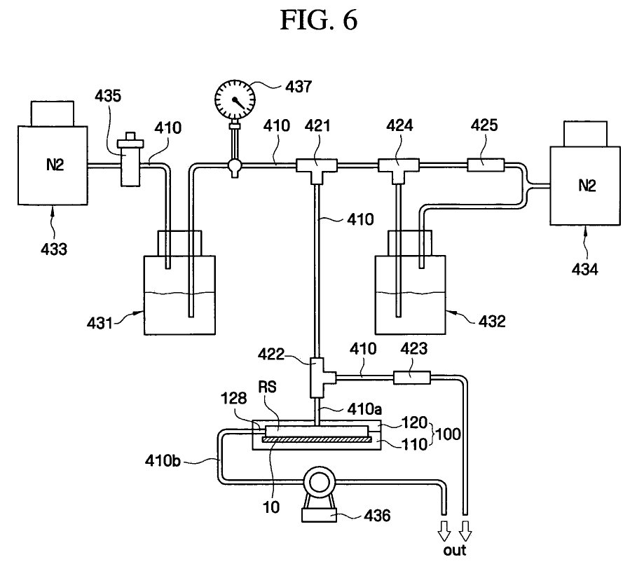

The flow of a fluid that is fed into or discharged from the reaction space RS is now described in more detail. FIG. 6 is a schematic diagram of a fluid flow system used for such flow of fluid(s) and/or a gas through the reaction space RS according to an example embodiment of the present invention.

The example fluid flow system of FIG. 6 includes a fluid inlet 410a, a fluid outlet 410b, first and second inactive gas supply tanks 433 and 434, respectively, and first and second sample tanks 431 and 432, respectively, a plurality of fluid flow tubes 410, and a plurality of valves 421, 422, 423, 424, and 425 connecting the fluid flow tubes.

The first and second sample supply tanks 431 and 432 store respective samples used for synthesis of biopolymers on the substrate 10. Such samples are supplied from the first and second sample supply tanks 431 and 432 to the reaction space RS within the reaction chamber 100 via the fluid inlet 410a.

Examples of a first biopolymer synthesis sample supplied from the first sample supply tank 431 include monomers such as a nucleoside, a nucleotide, an amino acid, or a peptide as described above and a compound thereof. For example, if oligonucleotide probes are in situ synthesized on the substrate 10, the biopolymer synthesis sample may be a nucleotide phophoramidite monomer having a base that is one of Adenine (A), Thymine (T), Guanine (G), Cytosine (c) and Uracil (U) and photolabile or acid labile protecting groups coupled thereto.

Examples of a second biopolymer synthesis sample supplied from the second sample supply tank 432 include a cleaning solution and an activator for activating synthesis of the aforementioned monomers. An activator for activating synthesis of phophoramidite monomers may be an acetonitrile solution, but is not limited thereto. The second sample supply tank 432 may be omitted in an alternative embodiment of the present invention. Alternatively, one second sample supply tank 432 may be connected with a plurality of fluid inlets 410a.

The first and second inactive gas supply tanks 433 and 434 each supply a respective inactive gas such as nitrogen (N₂) for example. The inactive gas from the first inactive gas supply tank 433 is supplied into the first sample supply tank 431 via the fluid flow tube 410 for applying pressure within the first sample supply tank 431. Such pressure within the first sample supply tank 431 causes the first biopolymer synthesis sample to be pushed up toward the fluid flow tube 410. The fluid flow system further includes a pressure controller 435 that is disposed between the first inactive gas supply tank 433 and the first sample supply tank 431 for adjusting the pressure there-between.

The inactive gas from the second inactive gas supply tank 434 is supplied into the second sample supply tank 432 via the fluid flow tube 410 for applying pressure within the second sample supply tank 432. Such pressure within the second sample supply tank 432 causes the second biopolymer synthesis sample to be pushed toward the fluid flow tube 410.

Furthermore, the inactive gas is supplied into the reaction space RS through the fluid flow tube 410 and the fluid inlet 410a without passing through the second sample supply tank 432 when the reaction space RS is maintained in an inactive state. Such inactive gas supplied into the reaction space RS prevents fluid flowing back from the reaction space RS into the fluid inlet 410a.

Each of the plurality of valves 421, 422, 423, 424, and 425 is a respective one of a 3-way solenoid valve or a 2-way solenoid valve. Referring to FIG. 6, each of first, second, and fourth valves 421, 422, and 424 is a respective 3-way solenoid valve, and each of third and fifth valves 423 and 425 is a respective 2-way solenoid valve.

Three terminals of the first valve 421 are respectively connected to the first sample supply tank 431, the second valve 422, and the fourth valve 424 through their corresponding fluid flow tubes 410. The fluid flow system further includes a pressure sensor 437 disposed between the first sample supply tank 431 and the first valve 421 along the fluid flow tube 410.

One of the three terminals of the second valve 422 is connected to the reaction space RS via the fluid inlet 410a. The other two terminals of the second valve 422 are respectively connected to the first and third valves 421 and 423 through the fluid flow tubes 410.

One terminal of the third valve 423 is connected to the second valve 422 through the fluid flow tube 410 while the other terminal is connected to a discharging opening (out). Three terminals of the fourth valve 424 are respectively connected to the first valve 421, the second sample supply tank 432, and the fifth valve 425 via the fluid flow tubes 410. Two terminals of the fifth valve 425 are connected to the fourth valve 424 and the second inactive gas supply tank 434 through the fluid flow tubes 410. The fluid outlet 410b has one end connected to the reaction space RS and the other end connected to the discharging portion (out) via a discharge pump 436.

An example of fluid flow operation within the fluid flow system of FIG. 6 is now described in more detail. First, the pressure controller 435 adjusts the pressure of the inactive gas supplied from the first inactive gas supply tank 433 to the first sample supply tank 431. Such inactive gas pressurizes the first biopolymer synthesis sample to be pushed up from the first inactive gas supply tank 433 towards the fluid flow tube 410 and then to reach the first valve 421.

In this case, if the first valve 421 is adjusted to block a passageway to the fourth valve 424 and to open a passageway to the second valve 422, the first biopolymer synthesis sample is fed into the second valve 422. If the second valve 422 is adjusted to create a passageway to the fluid inlet 410a, the first biopolymer synthesis sample is fed into the reaction space RS via the fluid inlet 410a.

Similarly for feeding the second biopolymer synthesis sample into the reaction space RS during or after supply of the first biopolymer synthesis sample into the reaction chamber, the inactive gas is fed into the second sample supply tank 432 from the second inactive gas supply tank 434. In that case, the fifth valve 425 is closed so that the inactive gas is fed into the second sample supply tank 432.

Such inactive gas pressurizes the second biopolymer synthesis sample to flow out from the second sample supply tank 432 towards the fourth valve 424 that is adjusted to create a passageway to the first valve 421. The first valve 421 is adjusted to create a passageway through the first valve to the second valve 422 for the second biopolymer synthesis sample. In addition, the second valve 422 is adjusted to create a passageway to the fluid inlet 410a such that the second biopolymer synthesis sample is fed into the reaction space RS via the fluid inlet 410a.

In an embodiment of the present invention, the inactive gas is fed into the reaction space RS during or after supply of the first and/or second biopolymer synthesis samples. For example, the fifth valve 425 is opened, and the fourth valve 424 is adjusted such that the inactive gas passes through only between the fifth and first valves 425 and 421. The second valve 422 is adjusted similarly as described for the second biopolymer synthesis sample such that the inactive gas is fed into the reaction space RS via the second valve 422 and the fluid inlet 410a.

Such inactive gas to the reaction space RS may be used to maintain the reaction space RS inactive. In addition, providing such inactive gas to the reaction space RS may effectively prevent the first and/or second biopolymer synthesis samples from flowing back into the fluid inlet 410a.

Furthermore, such inactive gas supplied to the reaction space RS results in creation of bubbles within the first and/or second biopolymer synthesis samples in the reaction space RS for improved miscibility and spreadability of the first and/or second biopolymer synthesis samples resulting in increased reaction yield. In an alternative embodiment of the present invention, the inactive gas may be fed into the reaction space RS for bubble generation through at least one separate fluid inlet apart from the fluid inlet 401a for feeding the first and/or second biopolymer synthesis samples.

The fluid flow tube 410 is cleaned using the inactive gas in the same manner as described above in the step of supplying the inactive gas. During cleaning, the second valve 422 is adjusted to block the inactive gas from the fluid inlet 410a while opening a passageway to the third valve 423.

In order to remove the fluid samples remaining after reaction within the reaction space RS, the discharge pump 436 operates to apply negative pressure to the reaction space RS. As a result, the remaining fluid samples are discharged into a discharging portion (out) through the fluid outlet 410b.

Operation of the shaking unit 200 in the biopolymer synthesis apparatus of FIG. 1 according to an embodiment of the present invention is now described. In the specification, the term “shaking” includes vibrations, swaying, reciprocating motion, rotary motion, rolling motion, and any other non-stop movement of the reaction chamber 100. When the reaction chamber 100 is shaken with a biopolymer synthesis sample fed into the reaction space RS, the biopolymer synthesis sample spreads evenly over the reaction space RS such that the biopolymer is uniformly synthesized over the entire surface of the substrate 10.

In particular, if the substrate 10 has uneven surfaces and the biopolymer synthesis sample has high viscosity and low spreadability, the shaking of the reaction chamber 100 by the shaking unit 200 improves reaction yield. In addition, such shaking of the reaction chamber 100 enables synthesis of the biopolymer on the substrate 10 with a small amount of the biopolymer synthesis sample because even such a small amount of the biopolymer synthesis sample may uniformly wet the surface of the substrate 10. A rolling motion is described herein as an example of the shaking operation by the shaking unit 200.

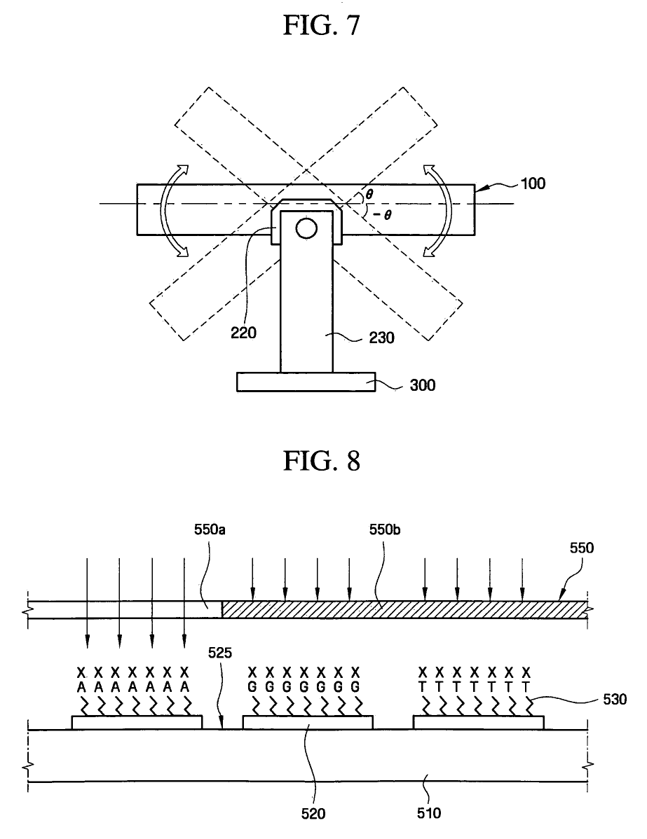

FIG. 7 is a side view illustrating shaking of the reaction chamber 100 by the shaking unit 200 according to an embodiment of the present invention. Referring to FIGS. 1, 2, and 7, the shaking unit 200 in the biopolymer synthesis apparatus of FIG. 1 according to an embodiment of the present invention includes a drive axis 220 and a servo motor 210 driving the drive axis 220.

The drive axis 220 has one end connected to the servo motor 210 and the other end connected to a support 230. The reaction chamber 100 is fixed onto the center of the drive axis 220. The servo motor 210 and the support 230 are disposed on a plate 300.

The servo motor 210 not only rotates the drive axis 220 but also causes the drive axis 220 to make a rolling motion with a predetermined period. The reaction chamber 100 fixed to the drive axis 220 also rotates or rolls along with the drive axis 220. The reaction chamber 100 may also rotate for discharging the biopolymer synthesis sample as will be described below. A maximum angle of rotation (+E in FIG. 7) by which the reaction chamber 100 rotates is ±90° in an example embodiment of the present invention, but the present invention is not limited thereto.

In an embodiment of the present invention, the reaction chamber 100 is rolled during synthesis of the biopolymer on the substrate 10 within the reaction space RS. The maximum angle ±θ that the reaction chamber rolls during such synthesis of the biopolymer varies depending on the amount of the biopolymer synthesis sample contained in the reaction space RS but is typically in a range of from about ±10° (i.e., the reaction chamber 100 rolling between −10° and +10°) and ±60° (i.e., the reaction chamber 100 rolling between −60° and +60°).

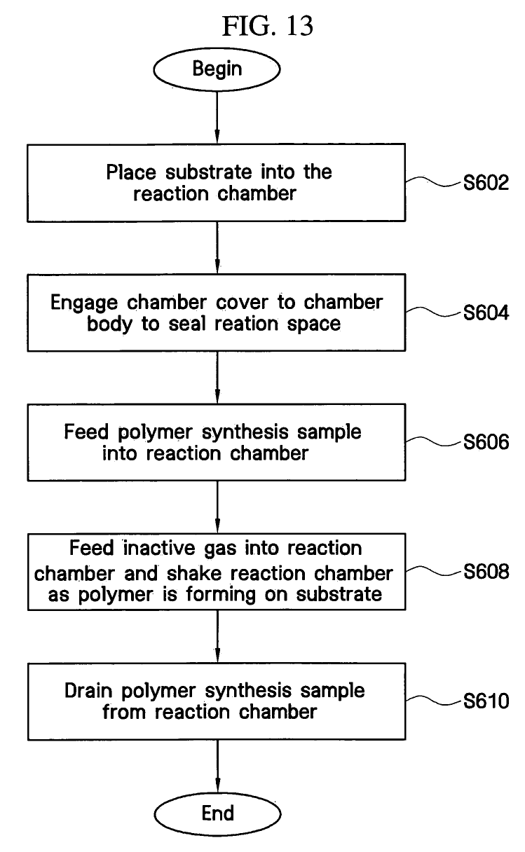

A method of synthesizing the biopolymer on the substrate 10 within the reaction space 10 according to an embodiment of the present invention is now described in more detail with reference to FIGS. 8, 9, 10, 11, and 13. FIG. 13 shows a flow-chart of steps performed during such synthesis of a biopolymer on the substrate 10 within the reaction space RS according to an embodiment of the present invention.

First, the substrate 10 is placed into the reaction chamber 100 (step S602 of FIG. 13). Subsequently, the chamber cover 120 is engageably combined with the chamber body 110 to seal the reaction space RS (step S604 of FIG. 13). Thereafter, at least one biopolymer synthesis sample is fed into the reaction space RS using the fluid flow system of FIG. 6 for example (step S606 of FIG. 13). In addition, the inactive gas is fed into the reaction space RS for creating bubbles in the biopolymer synthesis sample within the reaction space RS (step S608 of FIG. 13).

Furthermore, the reaction chamber 100 is shaken by the shaking unit 200 during formation of the biopolymer on the substrate 10 (step S608 of FIG. 13). Such shaking of the reaction chamber 100 ensures uniform spreading of the biopolymer synthesis sample over the substrate 10 for improving reactivity and yield for synthesis of the biopolymer on the substrate 10. After formation of the biopolymer on the substrate 10, the remaining biopolymer synthesis sample is drained from the reaction space RS (step S610 of FIG. 13).

FIGS. 8, 9, 10, and 11 show cross-sectional views illustrating the steps during synthesis of the biopolymer on the substrate 10 according to an embodiment of the present invention. Referring to FIG. 8, a substrate 510 (which may be the substrate 10 of FIGS. 4A and 4B) has formed thereon a plurality of cell active regions 520 and a plurality of cell separation regions 525 physically and/or chemically separating the plurality of cell active regions 520 from one another.

The cell active regions 520 may be formed of a silicon oxide layer such as plasma enhanced tetra-ethyl ortho silicate (PE-TEOS) layer, high-density plasma (HDP) oxide layer, P—SiH4 oxide layer, or thermal oxide layer, a silicate such as hafnium (Hf) silicate or zirconium (Zr) silicate, a metal oxynitride layer such as Si oxynitride layer, Hf oxynitride layer, or Zr oxynitride layer, a metal oxide layer such as titanium (Ti) oxide layer, tantalum (Ta) oxide layer, aluminum (Al) oxide layer, Hf oxide layer, Zr oxide layer, or indium tin oxide (ITO), a metal such as polyimide, polyamine, gold, silver, copper, or palladium (Pa), or a polymer such as polystyrene, polyacrylic acid, or polyvinyl.

Further in FIG. 8, each of the cell active regions 520 has a respective monomer formed thereon such as Adenine (A), Guanine (G), or Thymine (T) for example. However, the monomer may be a nucleotide phophoramidite monomer having a base that is any one of Adenine (A), Guanine (G), Thymine (T), Cytosine (C), or Uracil (U). Each of the plurality of monomers A, G, or T is coupled either directly or via a linker to the respective cell active region 520.

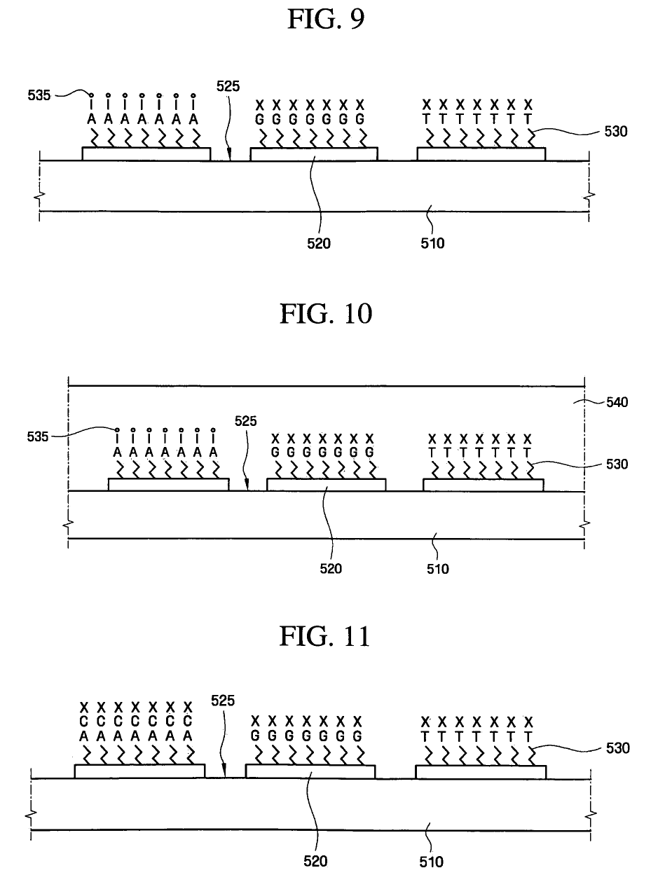

Each monomer A, G, or T contains a functional group (535 in FIG. 9) that may be coupled to another monomer and that is protected by a photolabile protecting group X. Examples of the functional group 535 may include hydroxyl, aldehyde, carboxyl, amide, thiol, halogen, or sulfonate groups.

Further referring to FIG. 8, a mask having a light transparent area 550a and a light blocking area 550b is placed over the substrate 510 for selectively exposing a predetermined one (having the monomer A in FIG. 8) of the cell active regions 520. From such exposure of light, the photolabile protecting group X coupled to the monomer A of the exposed cell active region 520 is removed for exposing the functional group 535 of the monomer A (as illustrated in FIG. 9).

FIGS. 10 and 11 illustrate the step of synthesizing the biopolymer on the substrate 510. Referring to FIG. 10, a biopolymer synthesis sample 540 is provided onto the resulting structure of FIG. 9 such as by being fed into the reaction space RS via the flow system of FIG. 6 for example. In the example of FIGS. 10 and 11, the biopolymer synthesis sample 540 is a nucleotide phophoramidite monomer CX having a base of Cytosine (C) protected by the photolabile protecting group X.

The biopolymer synthesis sample 540 selectively reacts with only the monomer A having the exposed functional group 535 that can be coupled to another monomer such as the nucleotide phophoramidite monomer CX in the biopolymer synthesis sample 540 to form the biopolymer ACX as illustrated in FIG. 11. The reaction chamber 100 having the substrate 510 therein is shaken by the shaking unit 200 during such formation of the biopolymer ACX for achieving uniform reaction across the entire substrate 510 resulting in improved reaction yield. Further referring to FIG. 11, the remaining biopolymer synthesis sample 540 is removed from the reaction space RS after formation of the biopolymer ACX.

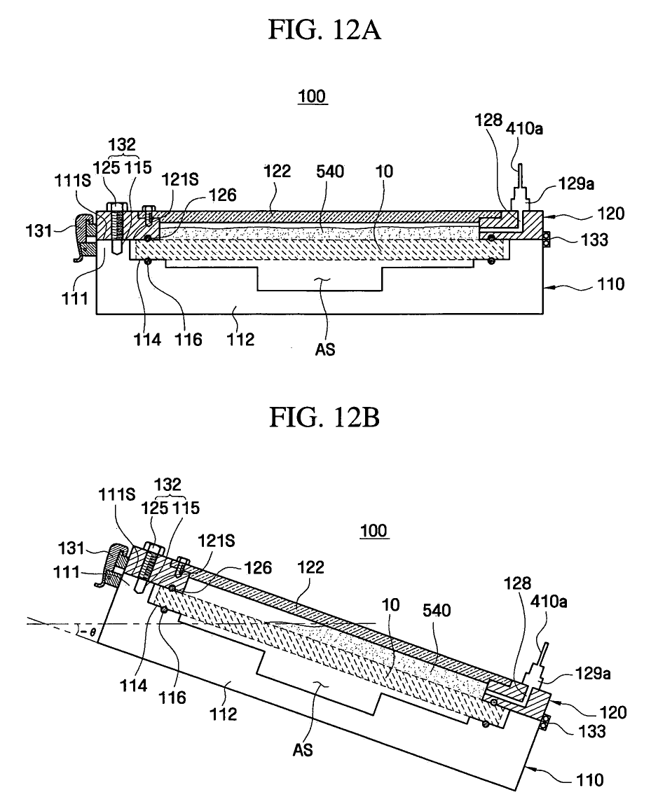

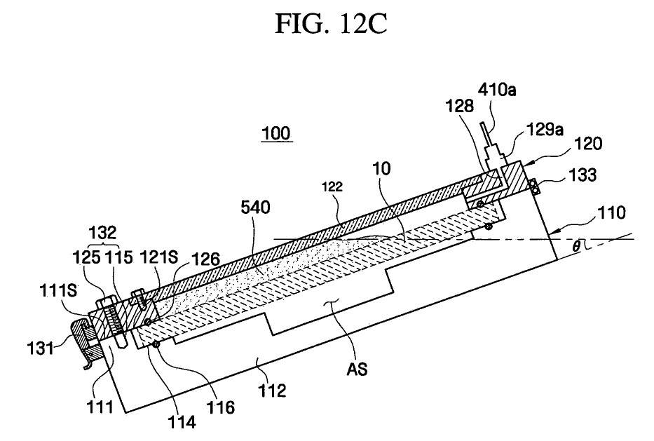

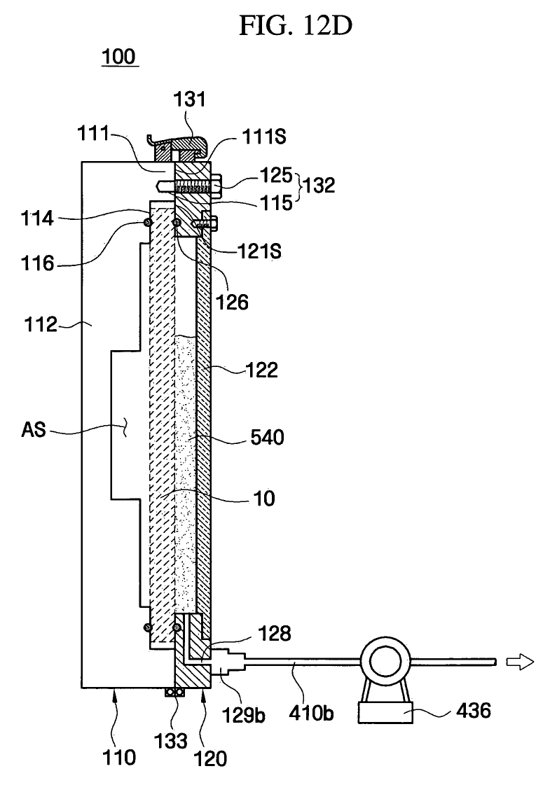

FIGS. 12A, 12B, 12C, and 12D are cross-sectional views illustrating an exemplary method for synthesizing the biopolymer on the substrate 10 within the biopolymer synthesis apparatus of FIG. 1, according to an embodiment of the present invention. Referring to FIG. 12A, the substrate 10 is placed on the substrate seating surface 114 of the chamber body 110. In addition, the first and second coupling units 131 and 132 are used to engage the chamber cover 120 to the chamber body 110. Thus, the edge of the substrate 10 is sealed by the rim 121 of the chamber cover 120 to form a substantially sealed reaction space RS between the substrate 10 and the chamber cover 120.

Thereafter, the biopolymer synthesis sample 540 is fed into the reaction space RS via the fluid inlet 410a, the connector 129a, and the through hole 128. The amount of the biopolymer synthesis sample 540 fed into the reaction space RS fills about 60% of the reaction space RS, in an embodiment of the present invention. However, the amount of the biopolymer synthesis sample 540 within the reaction space RS is not limited thereto and may be adjusted within a range of from about 10% to about 90% of the reaction space RS depending on the viscosity and the spreadability of the biopolymer synthesis sample 540 and on the conditions for subsequent shaking (velocity, time, and angle).

Additionally as described earlier, the substrate 10 has the edge sealed against the rim 121 of the chamber cover 120 and the substrate seating surface 114 of the chamber body 110 with the O-rings 116 and 126. Thus, the biopolymer synthesis sample 540 does not infiltrate into the air space AS for prevention of contamination of the rear surface of the substrate 10.

FIGS. 12B and 12C illustrate the step of rolling the reaction chamber 100 between the maximum angle of ±θ. By repeating the rolling motion between the angles of ±θ shown in FIGS. 12B and 12C, the biopolymer synthesis sample 540 spreads uniformly over the entire surface of the substrate 10, resulting in increased reaction yield. For example, if the maximum rolling angle is about ±30°, the reaction yield may be improved by about 30% compared to a reaction chamber that is not shaken.

Such rolling may cause the biopolymer synthesis sample 540 to flow back into the fluid inlet 410a. For preventing such backflow of the biopolymer synthesis sample 540, the inactive gas is fed into the fluid inlet 410a during such rolling. Such inactive gas applies pressure to the biopolymer synthesis sample 540 for suppressing or minimizing backflow of the biopolymer synthesis sample 540. If the reaction chamber 100 has a plurality of fluid inlets 410a, the inactive gas may be fed to all of the plurality of fluid inlets 410a.

Further, by feeding the inactive gas into the biopolymer synthesis sample 540 within the reaction space RS, bubbles are generated in the biopolymer synthesis sample 540 within the reaction space RS. Thus, the mobility and reactivity of the biopolymer synthesis sample 540 is further improved for increased reaction yield. The inactive gas may be supplied for such bubble generation independently of rolling of the reaction chamber 100. The inactive gas may be supplied via a separate fluid inlet dedicated for bubble generation instead of using the fluid inlet 401a.

FIG. 12D illustrates the step of discharging the biopolymer synthesis sample 540 remaining in the reaction space RS after formation of the biopolymer on the substrate 10. Referring to FIG. 12D, the reaction chamber 100 is rotated by 90° so that the fluid outlet 410b becomes located at the bottom. In addition, the discharge pump 436 operates to discharge the biopolymer synthesis sample 540 into a discharging portion via the through hole 128, the connector 129b, and the fluid outlet 410b.

If the reaction chamber 100 is rotated so that the fluid outlet is located at the bottom, discharge of the biopolymer synthesis sample 540 is further facilitated by gravity. Thus, the discharge pump 436 may operate with a low driving force. Alternatively, the biopolymer synthesis sample 540 may be discharged just by gravity without using the discharge pump 436. In that case, a valve is disposed at the fluid outlet 410b.

In this manner, the biopolymer synthesis apparatus and method according to embodiments of the present invention as described herein results in improved spreadability of the biopolymer synthesis sample and increased reaction yield. The biopolymer synthesis apparatus also prevents contamination of the rear surface of the substrate 10 without infiltration of the biopolymer synthesis sample into the air space from the reaction space RS.

While the present invention has been particularly shown and described with reference to exemplary embodiments thereof, it will be understood by those of ordinary skill in the art that various changes in form and details may be made therein without departing from the spirit and scope of the present invention as defined by the following claims. For example, the present invention may be used for forming any type of polymer on the substrate 10 using any type of polymer synthesis sample fed into the reaction space RS.

It is therefore desired that the present embodiments be considered in all respects as illustrative and not restrictive, reference being made to the appended claims rather than the foregoing description to indicate the scope of the invention. The present invention is limited only as defined in the following claims and equivalents thereof.

What is claimed is:

1. A method of synthesizing a polymer, the method comprising:
placing a substrate within a reaction chamber;
feeding a polymer synthesis sample into the reaction chamber for forming the polymer on the substrate; and
shaking the reaction chamber during formation of the polymer on the substrate within the reaction chamber.

2. The method of claim 1, wherein the reaction chamber includes a chamber body and a chamber cover that is combined with the chamber body to form a sealed reaction space.

3. The method of claim 2, further comprising:
sealing the reaction space with an edge of the substrate abutting a rim of one of the chamber body or the chamber cover.

4. The method of claim 3, wherein the reaction space is formed between the chamber cover and one surface of the substrate when the edge of the substrate abuts the rim of the chamber cover.

5. The method of claim 4, wherein an air space is formed between the chamber body and another surface of the substrate.

6. The method of claim 5, wherein the reaction space is sealed from the air space.

7. The method of claim 4, wherein another surface of the substrate abuts the chamber body.

8. The method of claim 4, wherein the chamber cover includes a transparent portion for viewing into the reaction space.

9. The method of claim 4, wherein the chamber cover includes at least one opening spatially coupled to at least one fluid inlet/outlet.

10. The method of claim 9, wherein the polymer synthesis sample is fed into the reaction space through the at least one opening.

11. The method of claim 10, further comprising:
feeding an inactive gas into the reaction chamber through the at least one fluid inlet/outlet for generating bubbles in the polymer synthesis sample within the reaction chamber during formation of the polymer.

12. The method of claim 11, wherein the polymer synthesis sample is drained through the at least one fluid inlet/outlet after formation of the polymer on the substrate.

13. The method of claim 4, wherein a distance of the reaction space between the chamber cover and the surface of the substrate is in a range of from about 0.2 mm (millimeters) to about 10 mm (millimeters).

14. The method of claim 3, wherein the reaction space is formed between the chamber body and one surface of the substrate.

15. The method of claim 2, wherein the polymer synthesis sample fills from about 10% to about 90% of the reaction space as the reaction chamber is shaken during formation of the polymer on the substrate.

16. The method of claim 15, wherein the polymer synthesis sample fills about 60% of the reaction space as the reaction chamber is shaken during formation of the polymer on the substrate.

17. The method of claim 1, further comprising:
feeding an inactive gas into the reaction chamber for generating bubbles in the polymer synthesis sample within the reaction chamber during formation of the polymer.

18. The method of claim 1, wherein the step of shaking of the reaction chamber includes:
rolling the reaction chamber with a maximum angle in a range of from about ±10° to about ±60°.

19. The method of claim 18, wherein the reaction chamber rolls with the maximum angle of about ±30°.

20. The method of claim 1, wherein the polymer is a biopolymer that is one of a nucleoside, a nucleotide, an amino acid, or a peptide, and wherein the substrate is one of a semiconductor wafer or a glass substrate.
